(19) United States
(12) Patent Application Publication
(10) Pub. No.: US 2011/0196212 A1
(43) Pub. Date: Aug. 11, 2011

(54) Methods and Systems for Health Wellness Management

(76) Inventors: John Peters, Cincinnati, OH (US); Gale Fogg, Cincinnati, OH (US)

(21) Appl. No.: 13/021,925

(22) Filed: Feb. 7, 2011

Related U.S. Application Data
(60) Provisional application No. 61/302,869, filed on Feb. 9, 2010.

(51) Int. Cl. A61B 5/00

(52) U.S. Cl. 600/300

(57) ABSTRACT

Systems and methods for health wellness management programs comprising a method of positively transforming behavioral and health parameters associated with members of a community and segmenting the community into attitudinal, behavioral and health parameter values. The method further includes enrolling members of the community into a health wellness management program, wherein each member pays a participation fee. The method may further include monitoring a progress status of each member of the community based on the collected behavioral and health parameter values. In addition, the method may further include environmental assessments (e.g., physical, social, cultural, economic) and programs for supporting adoption and maintenance of healthy behaviors within the community as a whole.

### CROSS REFERENCE TO RELATED APPLICATIONS

This application claims the benefit of U.S. Provisional Application No. 61/302,869, filed Feb. 9, 2010.

### TECHNICAL FIELD

Embodiments described herein generally relate to health improvement methods and systems and, more particularly, to health wellness management methods and systems tailored toward members of a community for achieving health goals of the community and its members.

### BACKGROUND

The rising prevalence of obesity and chronic disease (e.g., type 2 diabetes) in the population are a growing concern and are markers of unhealthy lifestyle behaviors. Today health care costs are rising at rapid rates. Organizations such as companies desire to both encourage their employees to practice a healthy lifestyle and to reduce their health care costs as much as possible. However, typical rates of participation in long-term voluntary wellness programs are low. Employees continue to practice unhealthy habits such as poor diet and lack of physical activity, in part because there are insufficient environmental supports for healthy behaviors and there are insufficiently powerful incentive and accountability systems for engaging people to change their behavior.

Accordingly, a need exists for alternative health wellness programs.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a graph illustrating overall percentage of weight loss of members enrolled in an exemplary health wellness management program according to one or more embodiments described herein; and

FIG. 2 is a graph illustrating cardiovascular fitness levels of members enrolled in an exemplary health wellness management program according to one or more embodiments described herein.

### DETAILED DESCRIPTION

Embodiments of the present disclosure are generally directed to business-to-business health wellness management methods and systems that empower members to succeed in long-term lifestyle changes. Although embodiments may be described herein as business-to-business health wellness management programs, embodiments are not limited thereto. For example, embodiments may be tailored to business-to-consumer health wellness management programs wherein the program is provided to individuals without the involvement of a community, such as a company or business entity.

Employee health wellness is the fastest growing segment of the employer delivered health benefit market. Embodiments described herein provide health and wellness information and training focused on delivering lasting lifestyle behavior change by applying approaches tailored to different community segments as found in a population of people. Community, as used herein, means a group of individuals associated with a particular organization, such as a business entity (e.g., a corporation), a religious institution, a government organization, a club, an educational institution, a geographical region, etc. Embodiments target the lifestyle behaviors of different segments of members within the community. Members are enrolled in a health wellness management program that has the greatest impact on prevention of chronic health conditions, e.g., obesity, cardiovascular disease and type 2 diabetes. Embodiments may also target environmental and social/cultural change elements that can support long term adoption of healthy behaviors by members.

In one embodiment, the health wellness management system is a transformational program that delivers lasting lifestyle change. The health wellness management methods and systems may be described herein as a program. The program is science based, holistic and personalized with a rewards and accountability structure that creates and sustains long-term engagement. The program comprises an introductory phase and a habituation phase. The introductory phase, which may be six months in duration, for example, is designed to introduce members to healthy fitness and diet information by focusing on teaching, tailoring and practicing new healthy lifestyle behaviors including nutrition and physical fitness. This introductory phase may be effectively free to members or may require a participation fee as described below. After completing the introductory phase, the members will enter the habituation phase, which may be for 18 months, for example, and is designed to provide members with the tools, techniques and information to maintain a healthy lifestyle for the rest of their lives. This second phase focuses on turning the newly learned behaviors into habits which involves mapping the behaviors to the individual's personal values and accountability systems. Embodiments focus on how to build a new life—weight loss and better health are side effects of building a new healthy lifestyle. It is understood that the introductory phase and the habituation phase may comprise other timeframes besides the examples provided above.

The heath wellness management methods and systems described herein may provide benefits to both employer and employee when implemented in the culture of a business entity. Although embodiments may be described in the context of a business entity having employer-employee relationships, embodiments are not limited thereto as described above. The health wellness management system may control health care costs by improving both direct (e.g., disease management) and indirect (e.g., disease avoidance, productivity improvements) measures of employee cost to the company. Rather than providing a health and fitness service to employees (i.e., members) that does not require any investment by the participant or requiring a constant fee for participation, the methods and systems described herein utilize a pay-for-performance methodology in which the employer pays a portion (or all) of the participation fee to employees that are enrolled in the program and have met certain health-related goals. The payfor-performance methodology provides that the employers pay for performance so that the associated employer costs for the program depends on the ability of the program to deliver particular outcomes. Under this model, the health wellness management system delivery cost is shared between the employees and the employer. The proportional contribution of the employer and enrolled employees is dependent on the outcome—the better the outcome, the more the employer pays of the cost of delivery while the employee pays more if his or her participation in the program does not produce the expected outcome by meeting certain health related goals. Employees will benefit in both short and long term measures such as weight loss, improved fitness and health markers, decreased medication need (for those with existing chronic conditions), energy level, self-esteem, etc. This is different from current programs that reward employees for participation rather than achieving specific results. Employers will benefit in a reduction in direct health care costs, reduced absenteeism related to health conditions, and potential for improved productivity (e.g., reduced presenteeism). Reduced direct health care costs may be achieved in a variety of ways. For example, an insurance provider may offer reduced premium rates, credits, insurance cost refunds, shared costs for implementing the health wellness program, etc. Any arrangement between the employer and an insurance company may be instituted based on implementation of the health wellness program and/or generation of successful results.

The pay-for-performance methodology may create a sense of ownership and accountability for those members of the community enrolled in the program, which may aid in maintaining retention rates among those enrolled. By paying participation fees upfront and having the ability to gain a reduction in such participation fees, members may be naturally biased to engage with the program for the long term. Behavioral economics suggests that when people feel invested in something, they desire to gain a return on their investment. For example, the theory of loss aversion provides that people will work toward achieving a goal in order to prevent the loss of something of value (e.g., a monetary sum, an object, etc.). Having participants commit to an extended program period is also an effective means of providing ongoing accountability to support long term behavior change.

In one embodiment, members pay a participation fee at regular intervals to remain enrolled in the program. Such intervals may be quarterly, monthly or other time frames. As an example and not a limitation, members may pay a monthly participation fee of $80.00 every month for access to the benefits of the system. The participation fee may be assessed to each member at the time of enrollment or after the introductory phase in which access to the system is free. In one embodiment, the introductory period is six months. If the introductory phase is free, participation fees may be assessed once the introductory phase is completed. As another example, members may pay all or a significant fraction of the total cost up front. As described in more detail below, if a member reaches one or more particular health related goals (e.g., a weight loss of 10%) after a particular period of time, such as at the end of the introductory phase or during the habituation phase (e.g., 12 months after enrollment), the member may receive a 50% rebate on the participation fee such that he or she may then pay a monthly participation fee of $40.00 for the remainder of the program. In another embodiment, the reward for meeting particular health related goals may be a lump sum payment at the end of the program, or some other predefined time period. The reward may be a rebate of 50% of the prepaid participation fees, for example. The rebate program or lump sum payment aids in program retention as the members will desire to achieve the reward in addition to practicing a more healthy lifestyle. The monthly payment serves as a periodic reminder of their investment and their commitment to achieving and maintaining a healthy lifestyle, and additionally serves as an element of accountability to help them succeed. Further, in one embodiment, the member may sign a contract such that the member is contractually obligated to complete the introductory phase, the habituation phase, or both. This long term commitment may add to the accountability structure.

Enrolled members are assigned to a particular segment of the community according to his or her health parameter values. Health parameter values may include a wide variety of health data, such as, for example, age, weight, body mass index, disease (e.g., diabetes, heart disease), stress level, cholesterol, etc. The community, which may include a company or a department within a company, for example, may be segmented based on the overall health parameter values of the members of the community, overall goals of the community (e.g., 75% percent of the enrolled members having a weight loss of more than 10%), behavioral economics personality, attitudes and beliefs about health and health behaviors, and historical data of the overall population. The program may segment the community by data that is collected about each of the members or a sample of the community. Data may be collected by the use of surveys and interviews, for example. The data for the particular community may be analyzed to segment the community into a plurality of segments, wherein each segment will have a particular consumer experience associated therewith. This approach is different than current one size fits all approaches, or approaches that simply target those who are the most unhealthy. The methods and systems described herein also provide consumer experiences for those who are healthy to ensure that they continue practicing a healthy lifestyle and delay significant disruptions to their health and their attendant healthcare costs. Custom tailored consumer experience based on segments may lead to enhanced participation and adherence.

Examples of types of segments will now be described. In one embodiment, the community may be segmented into four segments. A first segment may be the healthiest segment. As an example, members within this segment may have an average body mass index (BMI) of 25, and actively take care of themselves by eating well, exercising, seeking help for their well-being and reducing stress. Most members of this segment are optimistic and agree they can take steps to control their health. Most are likely to be interested in programs tailored toward maintaining and improving their health.

A second segment may include members that are generally healthy without requiring much effort (e.g., a BMI of 26). These members may tend to follow a healthy diet and exercise but do not focus on their health. The members in this group may be naturally healthy now but may become unhealthy without some changes to their diet and physical fitness levels. These members may need an incentive to maintain their health status.

A third segment may include members that are generally unhealthy (e.g., a BMI of 29) and critical of their body weight and are willing to make a change. The members of this segment may be concerned with their ability to follow through with achieving their goals. They may be interested in managing their health but often struggle to do so because of other commitments.

A fourth segment may include members that are the most unhealthy (e.g., a BMI of 32) and feel less in control of their health and are the least physically active. They are less likely to seek information about health and nutrition although they have a basic need to do so.

The exemplary segments described above may be dominated by a particular sex. For example, the third segment may have a higher percentage of women and the second segment may have a higher percentage of men. Therefore, the segments may be broken down further based on the sex of the members. A custom-tailored consumer experience may be provided for a particular sex within the segment. For example, a consumer experience may be designed and provided for women of the third segment. Consumer experiences may also be provided that are inclusive of both men and women. By breaking down the community into smaller segments of members having similar interests and health needs, a sense of cohesion among members may be developed within the segments that will enable the members to stay enrolled and to help one another meet their goals. For example, in an all women program within a particular segment, an “intelligent sorority” may be formed whereby the members support one another in achieving their goals.

After the community is segmented and members are placed in the appropriate segment and enrolled in a custom-tailored consumer experience, the members may be provided with information regarding their particular program. The information delivers a branded, holistic experience that is aspirational and relevant for life—it's not just about fixing a disease or delivering a short-term fad-like change in behavior. The consumer experience is configured and delivered like a desirable consumer product and not like a disease management program. The consumer experience offers comprehensive lifestyle skills training, integrating diet, nutrition, aerobic activity, strength training, lifestyle physical activity, environmental control, etc. all in one place as part of one approach.

Staff may be hired to provide health, diet and fitness information and training. Physical fitness trainers may be provided to deliver instruction to the members on how to properly implement lifestyle physical activity and planned exercise to meet their goals. Exercise and other physical activity sessions and opportunities may be provided at a work location or another location such as a local gym either during the work day or outside of working hours. The trainers will use the information provided to them by the health wellness management system to effectively convey the health related messages and advice. Nutritionists and dieticians or other qualified professionals may also be provided to deliver information on how to eat healthy and prepare healthy meals. Under the program, physicians or other qualified professionals may be invited to provide seminars on the human body, behavior change, lifestyle transformation, and ways to reduce disease risk.

Printed materials and electronic communications may be utilized to deliver the messages of the program to the members. A suite of tools may also be provided by monitoring the status and progress of the members in healthy lifestyle adoption and maintenance including, but not limited to, anthropometrics, metabolic and physical fitness measures, psychometric and behavioral measures.

The staff may work with the members to develop behavioral and health related goals that are custom-tailored for each member. The staff and members may discuss the member's readiness and environmental support to make the lifestyle change. Based on the member's readiness and environmental support, the individual member may be placed in a readiness state. The health related goals and consumer experience (i.e., fitness and diet program) may be further refined based on the readiness state, as well as historical data from previous members having a similar readiness state.

The behavioral and health related goals may be based on the environmental, behavioral and health parameters described above, or other considerations such as healthcare costs. For example, one health parameter may be body weight. The staff and the member may agree on a target weight, such as a 10% weight loss. Another example may be a behavioral parameter such as meeting a target of 9,000 walking steps taken each day. Another goal may be lowering cholesterol to a particular level, or improving physical fitness to a particular level. Any health parameter may be used for the health related goal and any behavior parameter may be used for the behavioral goal. A member may have one or more behavior and health related goals depending on the discussion between the member and the support staff. The goals may be utilized as benchmarks for the reward methodology described above. For example, if the health related goal of a particular member is a 10% weight loss, the member may receive a 50% participation fee rebate after a certain amount of time (e.g., after one year in the program) for every month he or she has maintained a 10% weight loss.

The staff may work with organizational leadership of the community to develop environmental (e.g., physical, social, cultural, economic) goals that are appropriate to support healthier behavior choices for all community members, including those enrolled in the program. The environmental goals may be based on the number of different elements of the behavioral environment that are changed, what measurable effect the changes had on behaviors in the population, cost-effectiveness of the changes for modifying population behavior, or other measures.

The support staff (e.g., trainers, dieticians, physicians, etc.) work together to individually deliver each member his or her respective consumer experience in the form of physical activity, diet advice, and the community environment of his or her peers that are also participating in the program. The level of support staff involvement will vary depending on the segment. For example, members within the first segment may require very little support staff involvement. The consumer experience for members within this first group may be simply access to the company gym and measurements of health parameters. On the other hand, members of the fourth segment described above, for example, may need significant support staff involvement. The support staff may provide training on how to use equipment, how to monitor a heart rate, calculate calories burned, how to prepare high quality food, help and/or assist in motivating and/or keeping members participating, etc.

Embodiments described herein accumulate comprehensive physiological, psychometric, social and environmental data in real time. This data may be used to drive personalized goal setting, program tailoring, problem solving, social and environmental support systems necessary to drive long term success within the program. The data that is collected may be used to monitor the progress of each member and track the member's activity and changes in health parameters. The support staff may review the collected data and provide feedback to the members accordingly. The individual consumer experiences provided to the members may be altered based on the data that is collected. The collected data may also be used to track the progress of the segments or community as a whole. The collected data may also be used to provide statistics, charts, graphs, etc. Absenteeism, presenteeism and impact on healthcare costs resulting from the program may also be monitored and calculated. Based on the collected and analyzed data, individual and group evaluations may be made to determine whether the goal(s) of the community have been met. The data may be used in a feedback loop where the goals, program, and support systems are tailored for future participants based on successful programs and outcomes of prior participants.

Once the member has reached his or her health related goal, or upon entry into the habituation phase, the program shifts into providing guidance and tools to the members to maintain a healthy lifestyle indefinitely. In this manner, each member may continue to practice a healthy lifestyle well after emerging from the program.

As an example and not a limitation, a test of one embodiment of the health wellness management program described above was performed over a six month period. The data described below is provided for illustrative purpose and is not intended to limit the embodiments described herein. For the test, a corporation was selected as a community. The test provided for 50 open slots for the program. After segmenting the community, the program was tailored toward women over thirty years of age fitting the health parameters of the third segment described above. Out of 978 eligible female employees, 440 applied for the 50 open slots, thereby illustrating an unmet need.

The selected members participated in the program and completed an introductory phase of six months. After six months, less than 10% withdrew from the program, which is indicative of a high retention rate. Three withdrew because of disability/retirement/resignation and one withdrew for personal reasons.

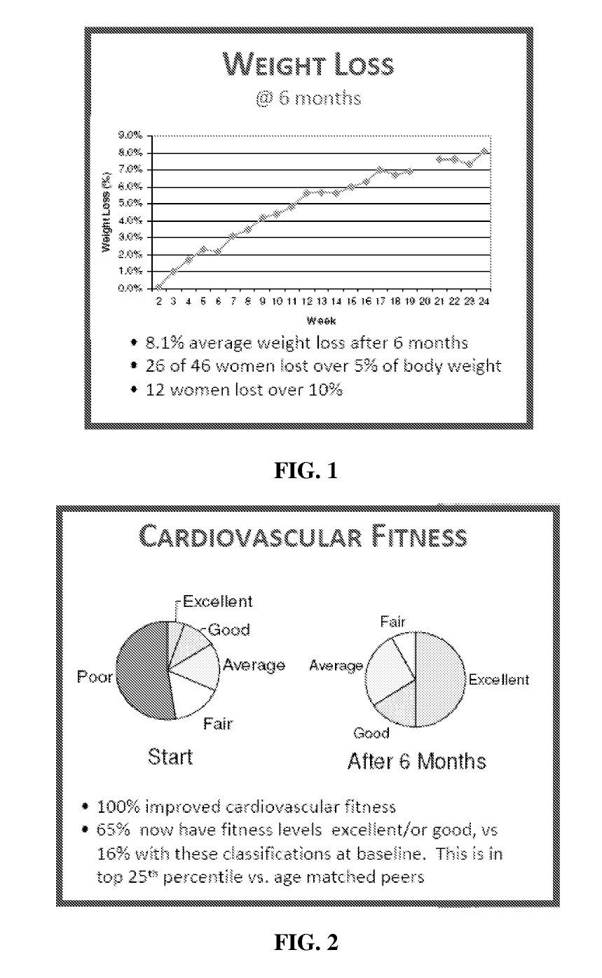

Results of the six month test are illustrated in FIGS. 1 and 2. After six months, more than one third of the women reduced their risk of type 2 diabetes, cardiovascular disease and hypertension. As shown in FIG. 1, the members enrolled in the program averaged 8.1% weight loss, with 26 of 46 women losing over 5% of body weight and 12 women losing over 10%. FIG. 2 shows that a majority of the women improved overall cardiovascular fitness and had an excellent or good fitness level by the end of the six month test. Additional metrics of data collected during the program are provided below in Table 1.

TABLE 1
AVERAGE METRICS FOR TEST PROGRAM

| | |
|---|---|
| Body Inches Lost | Key Health Measures |

| | | |
|---|---|---|
| Waist | −3.0 | Systolic BP dropped 3 mmHg (p < 0.03) |
| Hip | −3.1 | HDL increased by 14% (p < 0.0001) |
| Bust | −2.9 | Body fat decreased by 2.8% (p < 0.0001) |
| Thigh | −2.0 | BMI decreased 2.7 units 7.8% (p < 0.0001) |

Table 1 illustrates that the test health wellness management program enabled significant physical transformations of the test members, including, but not limited to, body size reduction and lower cholesterol parameter values. The physical transformations of the test members may translate into healthier and more active employees, as well as health reduction costs for the corporation. Test members indicated that the program enables a camaraderie that provides for an effective support system. At the completion of the introductory phase, more than 50% of the eligible test members signed an 18 month contract to complete the habituation phase at a cost of $79.99 per month with a 50% rebate provided if health related goals are met or continue to be met. It should be understood that the results and specific program details described in the above example are for illustrative purposes only.

Embodiments described herein may be offered as a kit for communities (e.g., organizations, business entities, etc.) to purchase and implement into the organizational environment. The kit may comprise a segmentation packet that includes information relating to segmenting the members of the community into one or more segments. For example, the segmentation packet may include software or survey questionnaires that asks individual members of a community certain questions (e.g., questions about health and fitness, questions about personality, etc.) to first develop segments and then place individuals into the appropriate segment. The segmentation packet may also provide information to an employer on the best practices for asking the segmentation questions, collecting the data, and segmenting the members.

The kit may also include a participation packet that provides information and instructions to the organization regarding participation fees, participation rewards, and program duration. Staff selection guidelines may also be provided that instruct the organization on what type of staff members to hire or contract, how to hire or contract staff, the minimum staff qualifications (e.g., certifications, etc.), expectations of staff members, etc.

An educational packet may be provided to the enrolled members. The educational packet includes tools related to health and fitness, and may include printed materials and electronic communication. Members may desire to subscribe to health newsletters or health alerts relating to their health status. Instructions regarding lifestyle physical activity, exercises, food preparation, stress reduction, and life skills may be provided as part of the program. Status monitoring tools, such as computer programs, may also be included in the educational packet to aid the members in monitoring health status and healthy lifestyle adoption. Information regarding long term preparation and goal setting while both in the program and outside of the program may also be provided.

A data collection tool may also be provided to the organization. The data collection tool may be a computer program that may be used to receive the various environmental, behavioral and health parameter data collected throughout the operation of the program. An analysis tool, which may be the same computer program as the data collection tool or a separate program, may be used to compile and display environmental, behavioral and health parameter data associated with members of the community. The analysis tool may also be configured to predict future performance of members based on historical data such that the program may be further refined. The analysis tool may monitor changes resulting from the program on an individual, segment, and/or community basis. Productivity, behavioral parameters, health parameters and healthcare costs may be monitored. The data collection tool may also comprise one or more computers (e.g., kiosks, desktops, laptops, personal computing devices, phones, combinations thereof, etc.) which may include the computer program and/or the analysis tool thereon to permit individuals, administrators, staff members, etc. to enter data and/or information and monitor and review this data and/or information as well as monitor and review the performance of individuals, segments, and/or communities.

An environmental assessment tool that measures the various physical, social, cultural and economic elements of the environment (e.g., at work, in a geopolitical community, etc.) that affect healthy lifestyle behavior choices may also be provided. This tool identifies specific elements of the environment that may be modified, and provides suggested approaches to better support adoption and maintenance of healthy lifestyle behaviors.

It should now be understood that embodiments described herein provide for systems, kits, and methods of health wellness management programs that have high enrollment and retention rates, that segment a community into specific segments, that tailor consumer experiences according to the segment and health parameters, establish goals and provide rewards when goals are met and maintained. Embodiments encourage and support a culture of well being in the workplace, as well as provide for a population level transformation in the rate of voluntary participation by the segmental targeting and customization of the program. Individual level transformations from an unhealthy to a healthy lifestyle may be provided by improved metabolic fitness, better physical fitness and improved self-efficacy.

Although the forgoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

Thus, many modifications and variations may be made in the techniques and structures described and illustrated herein without departing from the spirit and scope of the present claims.

Accordingly, it should be understood that the methods and apparatus described herein are illustrative only and are not limiting upon the scope of the claims.

It will be appreciated that any of the features, steps, or aspects of the present invention described herein may be combined, in whole or part, with any other feature, step, or aspect of the present invention described herein.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A kit for a health wellness management program comprising:
a segmentation packet comprising information relating to a method of segmenting members of a community into one or more segments;
a participation packet comprising information relating to participation, wherein information relating to participation comprises participation fees and program duration;
staff and facilities and equipment selection guidelines; facilities and equipment selection guidelines;
an educational packet for distribution among the members of the community, wherein the education information packet comprises information directed to at least physical fitness and diet;
a data collection tool in the form of a computer comprising a program for receiving environmental, behavioral and health parameter data associated with each member of the community and the community as a whole; and
an analysis tool in the form of a computer for compiling and displaying environmental, behavioral and health parameter data associated with the community.

2. A kit for a health wellness management program comprising:
a segmentation packet comprising instructions on how to segment members of a community into one or more segments;
a participation packet comprising participation fees and program duration;
staff, facilities and equipment selection guidelines comprising certification requirements;
an educational packet for distribution among the members of the community comprising at least physical fitness and diet instructions;
a data collection tool in the form of a computer comprising a program for receiving environmental, behavioral and health parameter data associated with each member of the community and the community as a whole; and
an analysis tool in the form of a computer for compiling and displaying environmental, behavioral and health parameter data associated with the community.

3. A method of positively transforming health parameters associated with members of a community comprising:
collecting data using a computer comprising a program to record environmental, behavioral and health parameter values associated with individual members of the community;
segmenting the community into one or more segments based at least in part on the environmental, behavioral and health parameter values, wherein members are associated with an appropriate segment based on their individual environmental, behavioral and health parameter values;
enrolling one or more members of the community into a health wellness management program, wherein each member pays a participation fee;
determining a physical activity program, a diet program, and one or more behavior and health parameter goals for each member of the community, wherein the physical activity program and diet program are determined based on at least the associated segment and the environmental, behavioral and health parameters values of the member;
determining an environmental modification program and one or more environmental parameter goals for the community as a whole;
providing training to each member of the community based at least in part on the determined physical activity program and diet program;
collecting with a computer comprising a program to record behavior and health parameter values for each member of the community;
monitoring a progress status of each member of the community based at least in part on the collected behavior and health parameter values;
providing a reward to each member of the community if the member reaches one or more behavior and health parameter goals after an introductory phase of the wellness program and upon successful achievement and maintenance of behavior and health parameter goals at one or more specified intervals throughout the program; and
evaluating a community goal based at least in part on the environmental, behavioral and health parameters of the members;
evaluating a community environmental change goal.

4. The method of claim 3 further comprising a habituation phase, wherein the habituation phase follows the introductory phase.
